(19) United States
(12) Patent Application Publication
(10) Pub. No.: US 2022/0047919 A1
(43) Pub. Date: Feb. 17, 2022

(54) INFORMATION PROCESSING APPARATUS, INFORMATION PROCESSING METHOD, AND NON-TRANSITORY STORAGE MEDIUM

(71) Applicant: Toyota Jidosha Kabushiki Kaisha, Toyota-shi Aichi-ken (JP)

(72) Inventors: Yurika Tanaka, Yokosuka-shi Kanagawa-ken (JP); Shuichi Sawada, Nagoya-shi Aichi-ken (JP); Shin Sakurada, Toyota-shi Aichi-ken (JP); Yasuhiro Baba, Kamo-gun Gifu-ken (JP); Shintaro Matsutani, Kariya-shi Aichi-ken (JP); Tomoya Makino, Kariya-shi Aichi-ken (JP)

(21) Appl. No.: 17/397,389

(22) Filed: Aug. 9, 2021

(30) Foreign Application Priority Data
Aug. 11, 2020 (JP) 2020-135575

(51) Int. Cl. A63B 24/00; A61B 5/11; G16H 20/30

(52) U.S. Cl. CPC A63B 24/0075; A61B 5/1118; A63B 2220/12; A63B 2024/0078; G16H 20/30

(57) ABSTRACT

An information processing apparatus disclosed has a controller. The controller obtains circumstances information defined as information relating to circumstances around the present location of a user. The controller determines a recommended exercise as a kind of exercise that is suitable for the circumstances around the present location of the user on the basis of the circumstances information obtained. Then, the controller sends suggestion information to a user's terminal used by the user. The suggestion information is information that suggests the user to do the recommended exercise at the present location of the user.

### CROSS-REFERENCE TO RELATED APPLICATIONS

This application claims the benefit of Japanese Patent Application No. 2020-135575, filed on Aug. 11, 2020, which is hereby incorporated by reference herein in its entirety.

### BACKGROUND

### Technical Field

The present disclosure relates to a technology for providing information to users.

### Description of the Related Art

There is known a car navigation system that suggests to a user moving by means other than a vehicle or a place where a user can do physical exercise during a spare time on the basis of a destination and a scheduled time of arrival at the destination set by the user (see, for example, Patent Literature 1 in the citation list below).

### CITATION LIST

### Patent Literature

Patent Literature 1: Japanese Patent Application Laid-Open No. 2018-194471

### SUMMARY

An object of this disclosure is to provide a technology that can contribute to improvement of user's health.

Disclosed herein is an information processing apparatus for providing information to a user. The information processing apparatus may comprise, for example, a controller including at least one processor, the controller being configured to execute the processing of:

obtaining circumstances information defined as information relating to circumstances around the present location of a user;

determining a recommended exercise as a kind of exercise that is suitable for the circumstances around the present location of the user on the basis of the circumstances information; and

sending suggestion information to a user's terminal used by the user, the suggestion information being information suggesting the user to do the recommended exercise at the present location of the user.

Also disclosed herein is an information processing method for providing information to a user. The information processing method may comprise, for example, the following steps of processing executed by a computer:

a first step of obtaining circumstances information defined as information relating to circumstances around the present location of a user;

a second step of determining a recommended exercise as a kind of exercise that is suitable for the circumstances around the present location of the user on the basis of the circumstances information; and

a third step of sending suggestion information to a user's terminal used by the user, the suggestion information being information suggesting the user to do the recommended exercise at the present location of the user.

Also disclosed herein is an information processing program that causes a computer to implement the above-described information processing method and a non-transitory storage medium in which this information processing program is stored in a computer-readable manner.

This disclosure can provide a technology that can contribute to improvement of user's health.

### BRIEF DESCRIPTION OF DRAWINGS

FIG. 1 is a diagram illustrating the general configuration of an information providing system.

FIG. 2 is a block diagram illustrating exemplary components included in the information providing system.

FIG. 3 illustrates an exemplary structure of a user information table.

FIG. 4 illustrates a data structure of an activity pattern.

FIG. 5 illustrates an exemplary structure of an exercise information table.

FIG. 6 is a flow chart of a process performed by a server apparatus.

FIG. 7 is a flow chart of the first process performed by the server apparatus to determine whether or not circumstances allow a user to do an exercise at his/her present location.

FIG. 8 is a flow chart of the second process performed by the server apparatus to determine whether or not circumstances allow the user to do an exercise at his/her present location.

FIG. 9 is a flow chart of the third process performed by the server apparatus to determine whether or not circumstances allow the user to do an exercise at his/her present location.

### DETAILED DESCRIPTION

The user's health may be improved by doing physical exercise during a short spare time, such as a break time in his/her workplace, a waiting time until the arrival of a train, or a spare time at his/her home. However, the user may not think of what kind of exercise he or she should do in a short time as mentioned above. If the user does a kind of exercise that is inappropriate in his/her circumstances, it may possibly cause inconvenience to people around him/her.

An information processing apparatus disclosed herein has a controller, which is configured to obtain circumstances information relating to the circumstances around the present location of the user. The circumstances information mentioned here includes, for example, at least one of information about an attribute of the place at which the user is presently located, information about the population density of a specific area including the present location of the user, and the present date and time. The attribute of the place is, for example, the name or the type of the place or facility in which the user is presently located, examples of which include home, workplace, school, store, public square, park, train station, train, bus stop, bus, and road. The term “specific area” mentioned above refers to an area around the user, which may be, for example, a circular area having a radius of several meters around the user at its center. After obtaining such circumstances information, the controller determines a recommended exercise on the basis of the circumstances information. The recommended exercise is a kind of exercise that is suitable for the circumstances around the present location of the user, in other words, a kind of exercise that the user can do fitly in the circumstances around his/her present location. More specifically, the recommended exercise is a kind of exercise that the user can do at his/her present location without causing inconvenience to people around him/her. Then, the controller sends information suggesting the user to do the recommended exercise at the present location to a terminal used by the user. This information will also be referred to as “suggestion information” hereinafter. The terminal used by the user will be referred to as “user's terminal”.

The information processing apparatus according to the present disclosure can suggest the user to do a kind of exercise (i.e. recommended exercise) that is suitable for the circumstances around the present location of the user. Thus, the user can know what kind of exercise he or she should to in a short spare time as mentioned above. Consequently, the user can do exercise in a short time. Since the exercise suggested to the user is an exercise that is suitable for the circumstances around the present location of the user, the user can do the exercise without causing inconvenience to people around him/her. Therefore, the user can improve his/her health appropriately.

The information processing apparatus according to the present disclosure may obtain an activity pattern of the user. The activity pattern of the user is data used to determine the activity conducted by the user at the present date and time. This data may be generated on the basis of a schedule of the user or a record of activities of the user in the past. The controller may determine whether or not the circumstances allow the user to do an exercise at his/her present location on the basis of the activity pattern and the circumstances information. For example, if it is conjectured based on the activity pattern, the attribute of the place in question, and the present date and time that the user is taking a rest or waiting for something at his/her present location, the controller may determine that the circumstances allow the user to do an exercise at his/her present location. Even in the case where it is conjectured that the user is taking a rest or waiting for something at his/her present location, if the population density of the specific area is high, the controller may determine that the circumstances do not allow the user to do an exercise at his/her present location. If it is conjectured based on the activity pattern, the attribute of the place, and the present date and time that the user is doing a work (e.g. job or study) at his/her present location, the controller may determine that the circumstances do not allow the user to do an exercise at his/her present location. If it is conjectured based on the activity pattern, the attribute of the place, and the present date and time that the user is eating a meal, taking a bath, or sleeping at his/her present location, the controller may determine that the circumstances do not allow the user to do an exercise at his/her present location. If it is conjectured based on the activity pattern, the attribute of the place, the population density of the specific area, and the present date and time that the user is moving by crowded public transportation means, the controller may determine that the circumstances do not allow the user to do an exercise at his/her present location. In the case where it is determined that the circumstances do not allow the user to do an exercise at his/her present location, the controller may suspend the processing of sending suggestion information. This prevents suggestion information from being sent to the user's terminal in circumstances that do not allow the user to do an exercise at his/her present location. Therefore, the user will not be bothered by suggestion information sent to the user's terminal at an inappropriate time.

The controller may determine as the recommended exercise a kind of exercise that can be done with smaller motions when the population density of the specific area is high than when it is low. This can prevent the recommended exercise done by the user at his/her present location from causing inconvenience to people around him/her with improved reliability.

In determining the recommended exercise mentioned above, the controller may obtain effect information defined as information about the effect of exercise that the user desires. Then, the controller may determine the recommended exercise on the basis of the effect information and the circumstances information. The effect information may be, for example, information about a part of the body in which the user desires to build up the muscle strength, or information about a part of the body that the user desires to get in shape. This enables the controller to suggest to the user a kind of exercise that will bring about the effect desired by the user as a recommended exercise.

### Embodiment

In the following, a specific embodiment of the technology disclosed herein will be described with reference to the drawings. It should be understood that the dimensions, materials, shapes, relative arrangements, and other features of the components that will be described in connection with the embodiment are not intended to limit the technical scope of this disclosure only to them, unless otherwise stated. What is described in the following as an embodiment is a case where the technology disclosed herein is applied to an information providing system.

(Outline of Information Processing System)

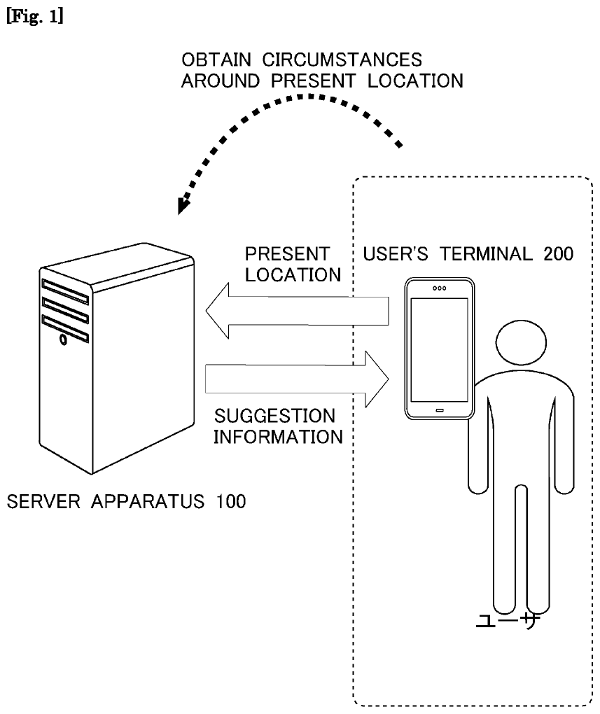

FIG. 1 is a diagram illustrating the general configuration of an information providing system. The information providing system includes a server apparatus 100 that provides suggestion information to users and user's terminals 200 used by the users. The suggestion information is information suggesting to a user a kind of exercise that the user can do at his/her present location.

The server apparatus 100 is an information processing apparatus that suggests to the user a kind of exercise that the user can do at his/her present location. The server apparatus 100 obtains information relating to the circumstances around the present location of the user (or circumstances information). The server apparatus 100 determines a recommended exercise on the basis of the circumstances information it obtained. The recommended exercise is a kind of exercise that is suitable for the circumstances around the present location of the user, in other words, a kind of exercise that the user can do without causing inconvenience to people around him/her. The server apparatus 100 creates information suggesting the recommended exercise determined as above to the user (i.e. suggestion information) and sends the suggestion information to the user's terminal 200.

The user's terminal 200 is a terminal used by the user. The user's terminal 200 may be, for example, a smartphone, a cellular phone, a tablet computer, or a wearable computer (e.g. smartwatch). The user's terminal 200 in the system of this embodiment has the functions of determining the present location of the user's terminal 200 (i.e. the present location of the user) and sending information about the determined present location (which will also be referred to as “location information” hereinafter) to the server apparatus 100. The user's terminal 200 sends the location information repeatedly at predetermined intervals (e.g. several minutes to a few hours).

(System Configuration)

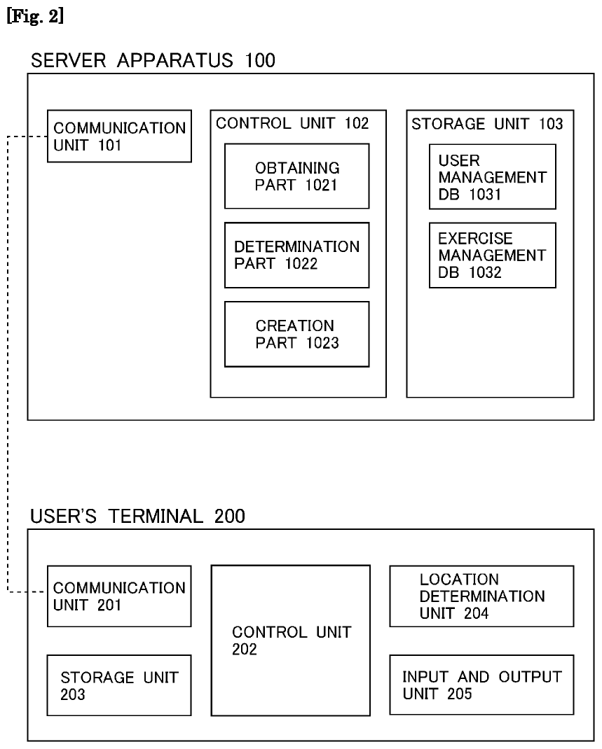

The configuration of the information providing system according to the embodiment will now be described with reference to FIG. 2. FIG. 2 is a block diagram schematically illustrating exemplary configurations of the server apparatus 100 and the user's terminal 200 shown in FIG. 1.

(Server Apparatus 100)

The server apparatus 100 is an information processing apparatus that suggests a recommended exercise to users. The server apparatus 100 may be constituted by a general-purpose computer. For example, the server apparatus 100 includes a processor, such as a CPU or a GPU, a main storage unit, such as a RAM or a ROM, and an auxiliary storage unit, such as an EPROM, a hard disk drive, or a removable medium. The removable medium may be a recording medium, such as a USB memory, a CD, or a DVD. The auxiliary storage unit stores an operating system (OS), programs, and tables. The processor executes a program(s) stored in the auxiliary storage unit to implement functions for achieving desired purposes that will be described later. Some or all the functions of the server apparatus 100 may be implemented by a hardware circuit(s), such as an ASIC or an FPGA.

As illustrated in FIG. 2, the server apparatus 100 of this embodiment includes a communication unit 101, a control unit 102, and a storage unit 103. The configuration of the server apparatus 100 is not limited to that illustrated in FIG. 2, but some components may be eliminated, replaced by other components, or added.

The communication unit 101 connects the server apparatus 100 to a network. For example, the communication unit 101 communicates with external apparatuses via the network using a communication network, such as LAN (Local Area Network), WAN (Wide Area Network), or Wi-Fi (registered trademark). The communication unit 101 communicates with the user's terminals 200 using a mobile communication service, such as 5G (5th Generation) mobile communications or LTE (Long Term Evolution) mobile communications, or a wireless communication network, such as Wi-Fi.

The control unit 102 is constituted by a processor, such as a CPU and performs overall control of the server apparatus 100. The control unit 102 of this embodiment has, as functional modules, an obtaining part 1021, a determination part 1022, and a creation part 1023. The control unit 102 implements these functional modules by executing a program stored in the storage unit 103 by the processor.

The obtaining part 1021 obtains circumstances information, that is, information relating to the circumstances around the user at his/her present location. The circumstances information in the system of this embodiment includes at least one of information about the attribute of the place in which the user is presently located (which will be referred to as “place in question” hereinafter), information about the population density of a specific area including the present location of the user, and the present date and time. In the process of obtaining the circumstances information, the obtaining part 1021 firstly obtains information about the attribute of the place in question and the present date and time. The attribute of the place is, for example, the name or the kind of the place or facility in which the user is presently located, examples of which include home, workplace, school, store, public square, park, train station, train, bus stop, bus, and road. In the case where the place in question is a place other than home, workplace, or school, the obtaining part 1021 obtains information about the population density of the specific area as circumstances information as well as information about the attribute of the place in question and the present date and time. The specific area is an area having a radius of several meters about the present location of the user in the place in question. The population density of the specific area is determined based on the number of other people located in the specific area. The number of other people located in the specific area may be determined based on location information received from terminals of them or an image captured by a camera or the like provided in the place in question.

The processing of obtaining circumstances information by the obtaining part 1021 is triggered by the reception of location information from the user's terminal 200 by the communication unit 101. After the completion of the processing of obtaining circumstances information, the obtaining part 1021 determines whether or not the circumstances allow the user to do an exercise at his/her present location. In making this determination, the obtaining part 1021 firstly obtains an activity pattern of the user. The activity pattern is data used to determine the activity of the user. The activity pattern is derived from data stored in the storage unit 103, which will be specifically described later. Then, the obtaining part 1021 determines whether or not the circumstances allow the user to do an exercise at the present location on the basis of the activity pattern of the user and the circumstances information.

If the place in question is the user's home and the activity of the user at the present date and time indicated by the activity pattern is an activity that the user cannot do while doing an exercise, such as eating a meal, taking a bath, or sleeping, the obtaining part 1021 may determine that the circumstances do not allow the user to do an exercise at the present location. If the place in question is the user's home and an activity of the user at the present date and time indicated by the activity pattern is an activity that the user can do easily while doing an exercise, such as an activity done during the user's leisure time (e.g. watching TV or movie or listening to music), the obtaining part 1021 may determine that the circumstances allow the user to do an exercise at the present location.

If the place in question is a workplace or school, and the activity of the user at the present date and time indicated by the activity pattern is working, studying, or eating a meal, the obtaining part 1021 may determine that the circumstances do not allow the user to do an exercise at the present location. If the place in question is a workplace or school, and the activity of the user at the present date and time indicated by the activity pattern is taking a rest, the obtaining part 1021 may determine that the circumstances allow the user to do an exercise at the present location.

If the user is waiting for a train or a bus at a train station or a bus stop, it is considered that the user has a time at his/her disposal until the arrival of the train or the bus. Nevertheless, if there are a relatively large number of other people around the user, it is probable that an exercise done by the user will cause inconvenience to other people around him/her. If the number of other people present around the user is relatively small, the possibility that an exercise done by the user causes inconvenience to other people around him/her is low. In view of this, if the place in question is a train station or a bus stop, the activity of the user at the present date and time indicated by the activity pattern is commuting or leisure, and the population density of the specific area is higher than a threshold, the obtaining part 1021 may determine that the circumstances do not allow the user to do an exercise at the present location. If the place in question is a train station or a bus stop, the activity of the user at the present date and time indicated by the activity pattern is commuting (to work or school) or leisure, and the population density of the specific area is lower than or equal to a threshold, the obtaining part 1021 may determine that the circumstances allow the user to do an exercise at the present location.

If the user is moving by public transportation means, such as a train or a bus, and the cabin of the public transportation means is not crowded, the possibility that an exercise done by the user with relatively small motions causes inconvenience to the other users around him/her is low. Therefore, if the place in question is a cabin of public transportation means, such as a train or a bus, the activity of the user at the present date and time indicated by the activity pattern is commuting (to work or school) or leisure, and the population density of the specific area is higher than a threshold, the obtaining part 1021 may determine that the circumstances do not allow the user to do an exercise at the present location. If the place in question is a cabin of public transportation means, the activity of the user at the present date and time indicated by the activity pattern is commuting (to work or school) or leisure, and the population density of the specific area is lower than or equal to the threshold, the obtaining part 1021 may determine that the circumstances allow the user to do an exercise at the present location.

When the user is waiting for friends etc. at an appointed place (e.g. a road, public square, park, or public space of a certain facility) other than the user's home, workplace, or school, it is considered that the user has a time at his/her disposal until the arrival of friends etc. Nevertheless, if there are a relatively large number of other people around the user, the possibility that an exercise done by the user causes inconvenience to other people around him/her is high. If the number of other people present around the user is relatively small, the possibility that an exercise done by the user causes inconvenience to other people around him/her is low. Therefore, if the place in question is a place other than the user's home, workplace, or school, the activity pattern at the present date and time of the user is waiting for friends etc., and the population density of the specific area is higher than a threshold, the obtaining part 1021 may determine that the circumstances do not allow the user to do an exercise at the present location. If the place in question is a place other than the user's home, workplace, or school, the activity pattern at the present date and time of the user is waiting for friends etc., and the population density of the specific area is lower than or equal to the threshold, the obtaining part 1021 may determine that the circumstances allow the user to do an exercise at the present location.

If it is determined that the circumstances do not allow the user to do an exercise at the present location, the server apparatus 100 does not provide suggestion information to the user. If it is determined that the circumstances allow the user to do an exercise at the present location, the server apparatus 100 provides suggestion information to the user. In the latter case, the circumstances information obtained by the obtaining part 1021 is passed from the obtaining part 1021 to the determination part 1022.

The determination part 1022 determines a recommended exercise on the basis of the circumstances information passed from the obtaining part 1021. As described previously, the recommended exercise is a type of exercise that is suitable for the circumstances around the present location of the user, in other words, an exercise that the user can do at his/her present location without causing inconvenience to people around him/her.

In the case where the place in question is the user's home, workplace, or school, it is considered that the user can do an exercise involving even relatively large motions without causing inconvenience to people around him/her. Therefore, if the place in question is the user's home, workplace, or school, the determination part 1022 may select a kind of exercise that matches properties (e.g. age, sex, or regular health problem) of the user as a recommended exercise from among a plurality of kinds of exercises including exercises involving relatively large motions. The kind of the recommended exercise may be changed every time it is sent from the server apparatus 100 to the user's terminal 200.

In the case where the place in question is a place other than the user's home, workplace, or school, the determination part 1022 determines a recommended exercise on the basis of the population density of the specific area in the place in question. For example, the determination part 1022 may select a kind of exercise that can be done only with smaller motions as a recommended exercise when the population density of the specific area is high than when it is low. In selecting a recommended exercise, the determination part 1022 may take into account the attribute of the place in question in addition to the population density of the specific area. For example, the determination part 1022 may select a kind of exercise that can be done only with smaller motions as a recommended exercise when the place in question is an indoor place (e.g. a store, a cabin of public transportation means, or a public space of a facility) than when the place in question is an outdoor place (e.g. a public square, a park, or a road). This allows the user to do an exercise without causing inconvenience to people around him/her or feeling embarrassed.

In determining the recommended exercise, the determination part 1022 may take into account the exercise effect that the user desires. In that case, information about the exercise effect that the user desires (i.e. effect information) may be stored in the storage unit 103, which will be specifically described later. The effect information may include, for example, information about a part of the body in which the user desires to build up the muscle strength, information about a part of the body that the user desires to get in shape, or information about a part of the body in which the user desires to increase flexibility. The determination part 1022 may determine the recommended exercise on the basis of the circumstances information passed from the obtaining part 1021 and the effect information stored in the storage unit 103. For example, the determination part 1022 may select a kind of exercise expected to provide the exercise effect that the user desires as the recommended exercise from among the exercises of the kinds that match the circumstances information.

The determination part 1022 may determine either one kind of recommended exercise or multiple kinds of recommended exercises. In the case where multiple kinds of recommended exercises are given, the user can select an exercise that he/she likes from among the multiple kinds of recommended exercises.

After the recommended exercise is determined as above, information about the recommended exercise is passed from the determination part 1022 to the creation part 1023.

The creation part 1023 creates suggestion information on the basis of the information about the recommended exercise passed from the determination part 1022. The suggestion information is information suggesting the user to do the recommended exercise at his/her present location. The suggestion information includes information indicating the kind of the recommended exercise, information about how to do the recommended exercise (i.e. method), or information about the effect of the recommended exercise. The suggestion information created by the creation part 1023 is sent to the user's terminal 200 through the communication unit 101.

The storage unit 103 stores various information. The storage unit is constituted by a storage medium, such as a RAM, a magnetic disk, or a flash memory. The storage unit 103 stores various programs executed by the processor and various data. Moreover, a user management database 1031 and an exercise management database 1032 are constructed in the storage unit 103 of this embodiment. These databases are constructed by managing data stored in the auxiliary storage unit by a database management system program (DBMS program) executed by the processor. The user management database 1031 and the exercise management database 1032 are, for example, relational databases.

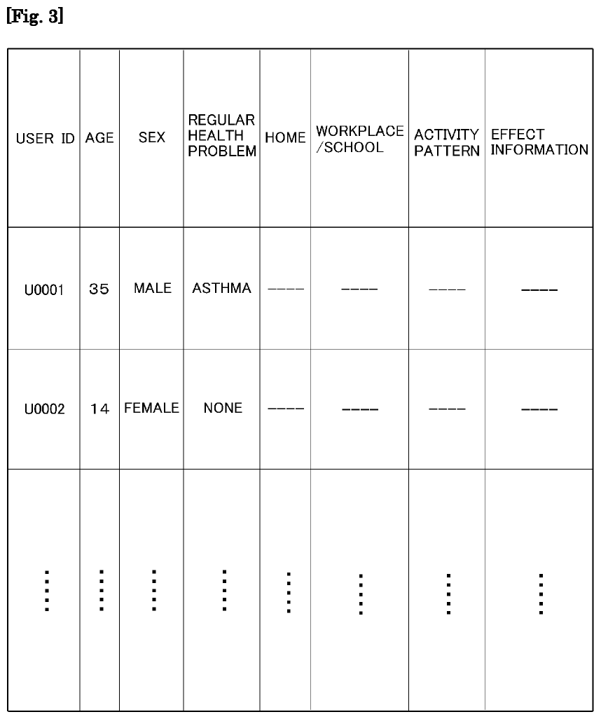

What is stored in the user management database 1031 is information about properties and activity patterns of the users who are under the management of the server apparatus 100. An example of the information stored in the user management database 1031 will be described with reference to FIG. 3. FIG. 3 illustrates the table structure of the information stored in the user management database 1031. As illustrated in FIG. 3, the table stored in the user management database 1031 has the fields of user ID, age, sex, regular health problem, home, workplace/school, activity pattern, and effect information. This table will also be referred to as the “user information table” hereinafter. The structure of the user information table is not limited to that illustrated in FIG. 3, but some fields may be added, changed, or removed fitly.

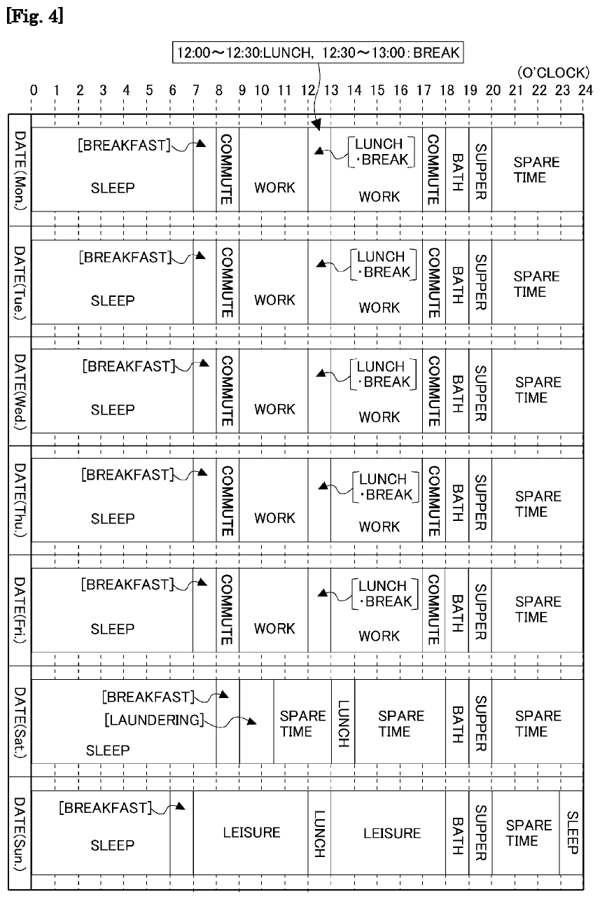

What is stored in the user ID field is information for identifying each of the users who are under the management of the server apparatus 100. This information will also be referred to as “user ID” hereinafter. What is stored in the age field is information indicating the age of each user. What is stored in the sex field is information indicating the sex of each user. What is stored in the regular health problem field is information indicating a regular health problem of each user. In the case where the user does not have any regular health problem, “NA” is stored in the regular health problem field. What is stored in the home field is information indicating the location of the home of each user (e.g. its address or latitude and longitude). What is stored in the workplace/school field is information indicating the location of the workplace or school to which each user commutes (e.g. its address or latitude and longitude). What is stored in the activity pattern field is an activity pattern of each user. As described previously, the activity pattern is data used to determine activities of the user. The activity pattern may be configured to describe, for example, a pattern of activities of the user through a week, as illustrated in FIG. 4. The data configuration of the activity pattern is not limited to that illustrated in FIG. 4, but it may be configured to describe a pattern of activities of the user through a month or a day. The activity pattern may be created on the basis of schedule information of each user or a record of activities of each user in the past. What is stored in the effect information field is information about an exercise effect that each user desires to achieve. Information stored in the effect information field may be, for example, “increasing jumping ability”, “flattening belly”, or “increasing flexibility of hip joint” etc. In the case where the user does not desire a particular exercise effect, “NA” is stored in the effect information field.

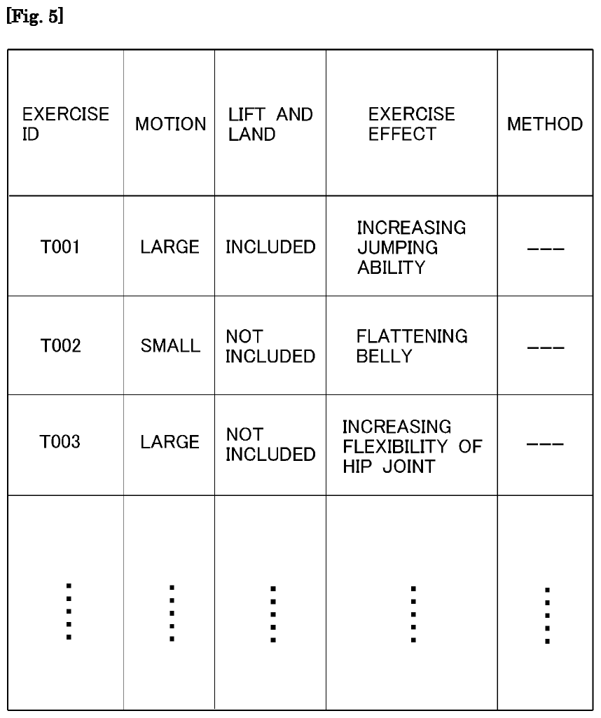

What is stored in the exercise management database 1032 is information about multiple kinds of exercises. An example of the information stored in the exercise management database 1032 will be described below with reference to FIG. 5. FIG. 5 illustrates the table structure of the information stored in the exercise management database 1032. As illustrated in FIG. 5, the table stored in the exercise management database 1032 has the fields of exercise ID, motion, lift and land, exercise effect, and method. This table will also be referred to as the “exercise information table” hereinafter. The structure of the exercise information table is not limited to that illustrated in FIG. 5, but some fields may be added, changed, or removed fitly.

What is stored in the exercise ID field is information for identifying each kind of exercise. This information will also be referred to as “exercise ID” hereinafter. The information stored in the exercise ID field may be the name of the exercise. What is stored in the motion field is information about the magnitude of motions involved in doing each exercise. While in the case illustrated in FIG. 5, the magnitudes of motions are classified into two classes of “large” and “small”, they may be classified into three or more classes. What is stored in the lift and land field is information indicating whether or not each exercise includes a motion of lifting and landing a foot from and on the floor or ground (e.g. stepping or jumping). For example, in the case of exercises including a motion of lifting and landing a foot from and on the floor, the information “included” is stored in the lift and land field. In the case of exercises not including a motion of lifting and landing a foot from and on the floor, the information “not included” is stored in the lift and land field. What is stored in the exercise effect field is information indicating an effect that is expected to be achieved when each kind of exercise is done. Examples of information stored in the exercise effect field may be “increasing jumping ability”, “flattening belly”, and “increasing flexibility of hip joint”. What is stored in the method field is information explaining how to do each kind of exercise.

Various processing executed by the server apparatus 100 configured as above may be executed by either hardware or software.

(User's Terminal 200)

Next, the user's terminal 200 will be described. The user's terminal 200 is a small computer carried by the user. As described above, the user's terminal 200 may be, for example, a smartphone, a cellular phone, a tablet computer, or a wearable computer (e.g. a smartwatch).

As illustrated in FIG. 2, the user's terminal 200 of this embodiment includes a communication unit 201, a control unit 202, a storage unit 203, a location determination unit 204, and input and output unit 205. The configuration of the user's terminal 200 is not limited to that illustrated in FIG. 2, but some components may be eliminated, replaced by other components, or added.

The communication unit 201 is a wireless communication interface that connects the user's terminal 200 to the network. The communication unit 201 communicates with the server apparatus 100 using, for example, a mobile communication service, such as 5G mobile communications or LTE mobile communications, or a wireless communication network, such as Wi-Fi (registered trademark).

The control unit 202 is constituted by, for example, a microcomputer and performs overall control of the user's terminal 200. For example, the control unit 202 controls a location determination unit 204 (which will be described later) to determine the present location of the user's terminal 200 (i.e. the present location of the user) at predetermined intervals. Every time the location determination unit 204 determines the present location, the control unit 202 sends location information to the server apparatus 100 through the communication unit 201. The location information includes information indicating the present location determined by the location determination unit 204 as above. The location information created by the user's terminal 200 of this embodiment also contains the user ID of the user who uses the user's terminal 200 besides the information indicating present location. The processing of determining the present location and the processing of sending the location information may be performed in response to a request made by the server apparatus 100.

The storage unit 203 stores various information. The storage unit 203 is constituted by a storage medium, such as a RAM, a magnetic disk, or a flash memory. The storage unit 203 stores various programs executed by the control unit 202 and various data. The storage unit 203 of this embodiment also stores a user ID of the user who uses the user's terminal 200.

The location determination unit 204 determines the location of the user's terminal 200. The location determination unit 204 typically includes a GPS receiver.

The input and output unit 205 is configured to receive inputting operations conducted by the user and present information to the user. The input and output unit 205 includes, for example, a touch panel, a control circuit for the touch panel, a liquid crystal display, a control circuit for the liquid crystal display, a microphone, a control circuit for the microphone, a speaker, and a control circuit for the speaker. The touch panel and the liquid crystal display may be a single touch panel display. The input and output unit 205 of this embodiment outputs or displays suggestion information sent from the server apparatus 100 through the touch panel display.

Various processing executed by the user's terminal 200 configured as above may be executed by either hardware or software.

(Process Performed by Server Apparatus)

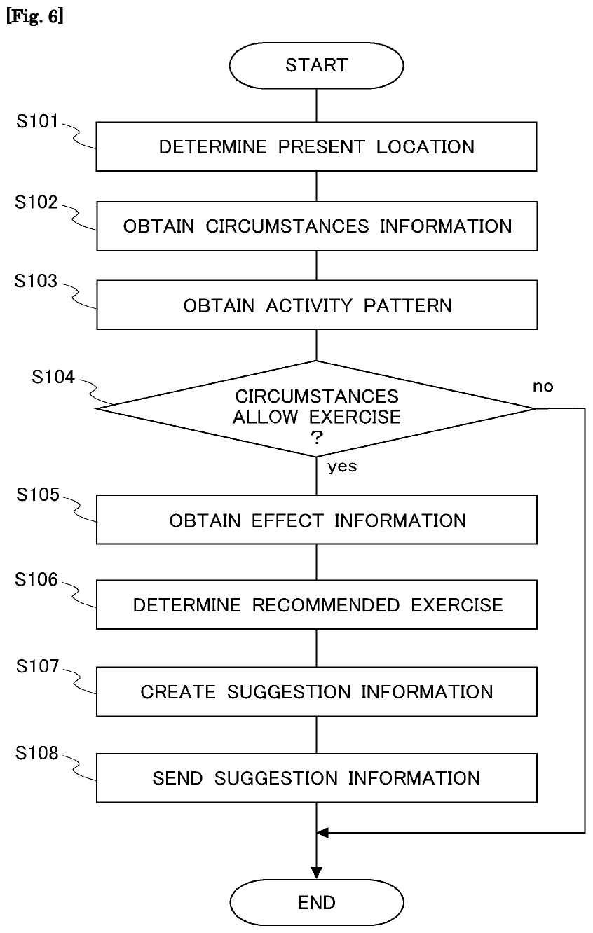

A process performed by the server apparatus 100 will be described next with reference to FIG. 6. FIG. 6 is a flow chart of the process performed by the server apparatus 100, which is triggered by reception of location information sent from the user's terminal 200.

In the process according to the flow chart of FIG. 6, when the communication unit 101 of the server apparatus 100 receives location information sent from the user's terminal 200, the obtaining part 1021 extracts the user ID and information indicating the present location from the location information to obtain the present location of the user (step S101).

The obtaining part 1021 obtains circumstances information on the basis of the present location of the user (step S102). Specifically, the obtaining part 1021 accesses the user management database 1031 using the user ID as an argument to find the user information table associated with the user. Then, the obtaining part 1021 compares the present location obtained in step S101 and the information stored in the home field and the workplace/school field of the user information table found as above to determine whether or not the place where the user is presently located (i.e. the place in question) is the user's home, workplace or school. If the place in question is the user's home, work place, or school, the obtaining part 1021 obtains information indicating that the attribute of the place in question is the user's home, workplace, or school and information indicating the present date and time as circumstances information. If the place in question is not the user's home, work place, or school, the obtaining part 1021 compares the present location of the user and map information to determine the attribute of the place in question. Moreover, the obtaining part 1021 obtains the population density of the specific area in the place in question. Then, the obtaining part 1021 obtains information indicating the attribute of the place in question, information indicating the population density of the specific area, and information indicating the present date and time as circumstances information.

The obtaining part 1021 also obtains the activity pattern of the user (step S103). Specifically, the obtaining part 1021 obtains the activity pattern of the user by retrieving the activity pattern stored in the activity pattern field of the user information table found in step S102.

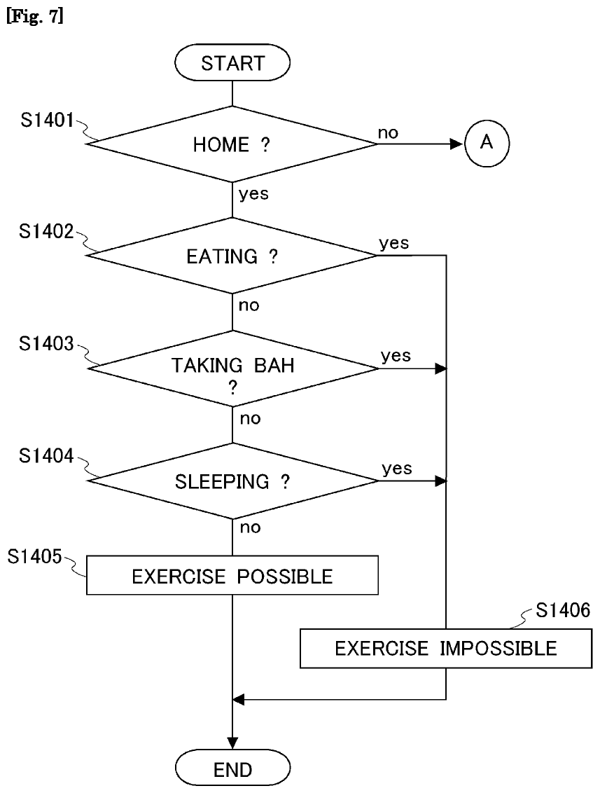

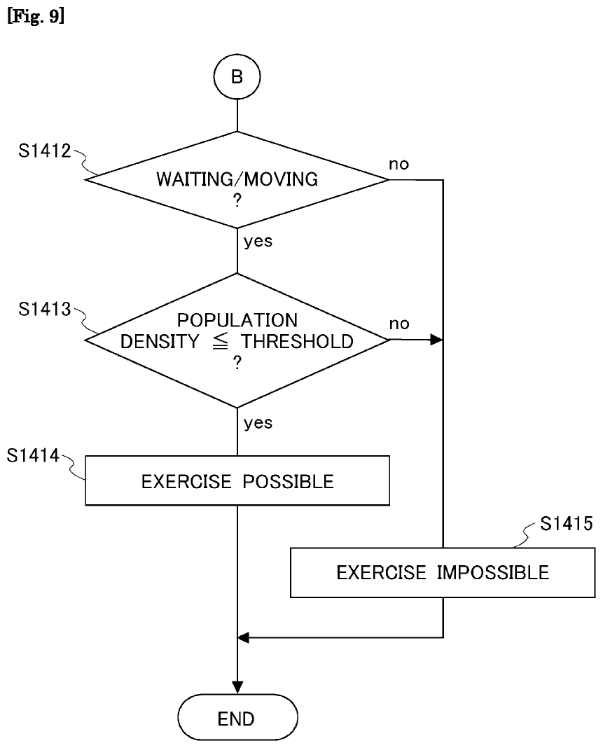

Then, the obtaining part 1021 determines whether or not the circumstances allow the user to do an exercise at the present location on the basis of the circumstances information obtained in step S102 and the activity pattern obtained in step S103 (step S104). The processing executed in step S104 will be specifically described in the following with reference to FIGS. 7 to 9.

As illustrated in FIG. 7, the obtaining part 1021 firstly determines whether or not the place in question is the user's home (step S1401). If the place in question is the user's home (an affirmative answer in step S1401), the obtaining part 1021 compares the present date and time with the activity pattern to determine whether or not the user is eating a meal (step S1402). Specifically, if the present date and time falls in a time slot of meal (breakfast, lunch, or supper) in the activity pattern like that shown in FIG. 4, the obtaining part 1021 determines that the user is eating a meal (an affirmative answer in step S1402). If the present date and time does not fall in a time slot of meal in the activity pattern, the obtaining part 1021 determines that the user is not eating a meal (a negative answer in step S1402).

If step S1402 is answered in the affirmative, the obtaining part 1021 determines that the circumstances do not allow the user to do an exercise at the present location (namely, exercise is impossible) (step S1406). If step S1402 is answered in the negative, then the obtaining part 1021 determines whether or not the user is taking a bath (step S1403). Specifically, if the present date and time falls in a time slot of bath in the activity pattern, the obtaining part 1021 determines that the user is taking a bath (an affirmative answer in step S1403). If the present date and time does not fall in a time slot of bath in the activity pattern, the obtaining part 1021 determines that the user is not taking a bath (a negative answer in step S1403).

If step S1403 is answered in the affirmative, the obtaining part 1021 determines that the circumstances do not allow the user to do an exercise at the present location (namely, exercise is impossible) (step S1406). If step S1403 is answered in the negative, then the obtaining part 1021 determines whether or not the user is sleeping (step S1404). Specifically, if the present date and time falls in a time slot of sleep in the activity pattern, the obtaining part 1021 determines that the user is sleeping (an affirmative answer in step S1404). If the present date and time does not fall in a time slot of sleep in the activity pattern, the obtaining part 1021 determines that the user is not sleeping (a negative answer in step S1404).

If step S1404 is answered in the affirmative, the obtaining part 1021 determines that the circumstances do not allow the user to do an exercise at the present location (namely, exercise is impossible) (step S1406). If step S1404 is answered in the negative, the obtaining part 1021 determines that the circumstances allow the user to do an exercise at the present location (step S1405).

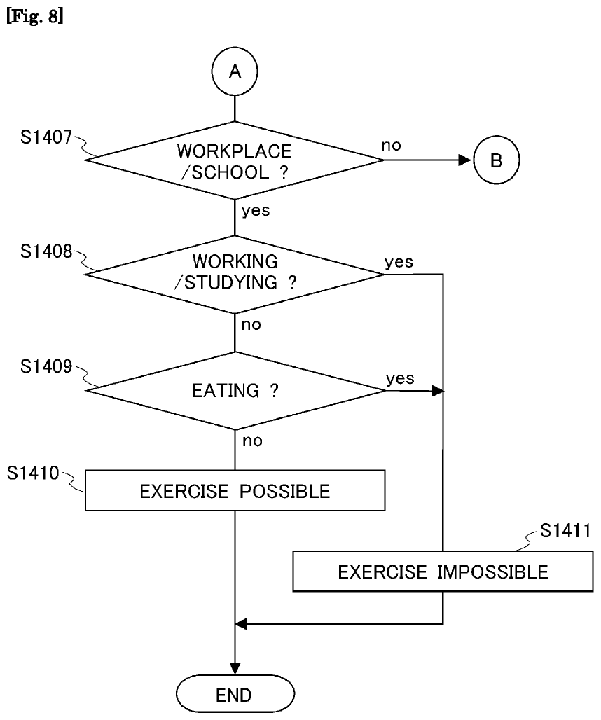

If step S1401 is answered in the negative, the obtaining part 1021 determines whether or not the attribute of the place in question is workplace or school (step S1407 in FIG. 8). If the attribute of the place in question is workplace or school (an affirmative answer in step S1407), the obtaining part 1021 compares the present date and time with the activity pattern to determine whether or not the user is working or studying (step S1408). Specifically, if the present date and time falls in a time slot of work or study in the activity pattern, the obtaining part 1021 determines that the user is working or studying (an affirmative answer in step S1408). If the present date and time does not fall in a time slot of work or study in the activity pattern, the obtaining part 1021 determines that the user is not working or studying (a negative answer in step S1408).

If step S1408 is answered in the affirmative, the obtaining part 1021 determines that the circumstances do not allow the user to do an exercise at the present location (namely, exercise is impossible) (step S1411). If step S1408 is answered in the negative, then the obtaining part 1021 determines whether or not the user is eating a meal (step S1409). Specifically, if the present date and time falls in a time slot of meal (lunch) in the activity pattern, the obtaining part 1021 determines that the user is eating a meal (an affirmative answer in step S1409). If the present date and time does not fall in a time slot of meal in the activity pattern, the obtaining part 1021 determines that the user is not eating a meal (a negative answer in step S1409).

If step S1409 is answered in the affirmative, the obtaining part 1021 determines that the circumstances do not allow the user to do an exercise at the present location (namely, exercise is impossible) (step S1411). If step S1409 is answered in the negative, the obtaining part 1021 determines that the circumstances allow the user to do an exercise at the present location (step S1410).

If step S1407 is answered in the negative, the obtaining part 1021 determines whether or not the user is waiting for something at the present location or moving (step S1412). Specifically, if one of the following two conditions is met, the obtaining part 1021 determines that the user is waiting for something at the present location or moving (an affirmative answer in step S1412). If neither of the following two conditions is met, the obtaining part 1021 determines that the user is not waiting something at the present location nor moving (a negative answer in step S1412).
Condition 1: The place in question is not a cabin of public transportation means, and the present date and time falls in a time slot of commute, leisure, or waiting.
Condition 2: The place in question is a cabin of public transportation means, and the present date and time falls in a time slot of commute or leisure.

If step S1412 is answered in the negative, the obtaining part 1021 determines that the circumstances do not allow the user to do an exercise at the present location (namely, exercise is impossible) (step S1415). If step S1412 is answered in the affirmative, the obtaining part 1021 determines whether or not the population density of the specific area is lower than or equal to a threshold (step S1413). This threshold is, for example, a value of the population density of the specific area below which it is considered that doing an exercise involving only minimum motions will not cause inconvenience to people around the user. If the population density of the specific area is lower than or equal to the threshold (an affirmative answer in step S1413), the obtaining part 1021 determines that the circumstances allow the user to do an exercise at the present location (step S1414). If the population density of the specific area is higher than the threshold (a negative answer in step S1413), the obtaining part 1021 determines that the circumstances do not allow the user to do an exercise at the present location (step S1415).

Referring back to the flow chart of FIG. 6, if step S104 is answered in the negative, the execution of this routine is ended. Then, the server apparatus 100 does not send suggestion information to the user's terminal 200. If step S104 is answered in the affirmative, the user ID of the user and the circumstances information obtained in step S102 are passed from the obtaining part 1021 to the determination part 1022.

The determination part 1022 accesses the user management database 1031 using the user ID passed from the obtaining part 1021 as an argument to obtain the effect information associated with the user (step S105). In other words, the determination part 1022 retrieves the information stored in the effect field of the user information table associated with the user.

Then, the determination part 1022 determines a recommended exercise on the basis of the circumstances information passed from the obtaining part 1021 and the effect information of the user (step S106). Specifically, the determination part 1022 accesses the exercise management database 1032 using the effect information as an argument to pick out all the kinds of exercises that match the effect information. Then, the determination part 1022 selects a kind of exercise that matches the circumstances information from among the picked-out exercises and determines this exercise as the recommended exercise. For example, in the case where the place in question is the user's home, workplace, or school, the determination part 1022 may select the exercise with the highest exercise effect that matches the effect information among the picked-out exercises as the recommended exercise. In the case where the place in question is the user's home that is an apartment, the determination part 1022 may determine the recommended exercise taking into account whether a resident of a neighboring apartment or a resident of the apartment immediately below the user's apartment is at home or not. For example, if a resident of a neighboring apartment or a resident of the apartment immediately below the user's apartment is at home, the determination part 1022 may select a kind of exercise without a motion of lifting and landing a foot from and on the floor among the exercises having an exercise effect that matches the effect information as the recommended exercise. In the case where the place in question is a place other than the user's home, workplace, or school, the determination part 1022 may select a kind of exercise having an exercise effect that matches the effect information that can be done only with motions whose magnitude is allowable for the population density of the specific area as the recommended exercise. In the case where the information stored in the effect information field of the user information table associated with the user is “NA”, the determination part 1022 may determine a recommended exercise on the basis of properties (e.g. age, sex, or regular health problem) of the user and the circumstances information. After determining the recommended exercise as above, the determination part 1022 passes the exercise ID of the recommended exercise to the creation part 1023.

The creation part 1023 creates suggestion information on the basis of the information passed from the determination part 1022 (step S107). As described previously, the suggestion information is information suggesting the user to do the recommended exercise at his/her present location. The suggestion information contains information indicating the kind of the recommended exercise, information explaining how to do the recommended exercise, and information indicating the exercise effect of the recommended exercise. The information explain how to do the recommended exercise and the information indicating the exercise effect of the recommended exercise are retrieved from the exercise information table associated with the recommended exercise. The suggestion information created by the creation part 1023 is sent to the user's terminal 200 through the communication unit 101 (step S108). The user's terminal 200 receives this suggestion information, and its control unit 202 causes the input and output unit 205 to output the suggestion information.

The processing routine according to the flow charts of FIGS. 6 to 9 can suggest to the user a kind of exercise that is suitable for the circumstances around the present location of the user. Therefore, the user can know what kind of exercise he or she should do in a short time, such as a break time in the workplace, a waiting time until the arrival of a train, or a spare time at his/her home. In consequence, the user can do an exercise using a short spare time as mentioned above. Since the exercise suggested to the user is an exercise that is suitable for the circumstances around the present location of the user, the user can do the exercise without causing inconvenience to people around him/her. Moreover, a kind of information that matches the effect information is suggested to the user. Therefore, the user can achieve a desired exercise effect. When the circumstances do not allow the user to do an exercise at his/her present location, the server apparatus 100 does not send suggestion information to the user's terminal 200, so that the user is not bothered.

As above, the information providing system according to the above embodiment can improve the user's health favorably.

### <Others>

The above embodiment has been described only by way of example. Modifications can be made to them without departing from the essence of this disclosure. One, some, or all of the processes that have been described as processes performed by one apparatus may be performed by a plurality of apparatuses in a distributed manner. One, some, or all of the processes that have been described as processes performed by different apparatuses may be performed by a single apparatus. The hardware configuration employed to implement various functions in a computer system may be modified flexibly.

The technology disclosed herein can be carried out by supplying a computer program(s) that implements the functions described in the above description of the embodiment to a computer to cause one or more processors of the computer to read and execute the program(s). Such a computer program(s) may be supplied to the computer by a computer readable, non-transitory storage medium that can be connected to a system bus of the computer, or through a network. The computer-readable, non-transitory storage medium refers to a recording medium that can store information, such as data and programs, electrically, magnetically, optically, mechanically, or chemically in such a way as to allow the computer or the like to read the stored information. Examples of such a recording medium include any type of disc media including a magnetic disc, such as a floppy disc (registered trademark) and a hard disk drive (HDD), and an optical disc, such as a CD-ROM, a DVD, and a Blu-ray disc. The recording medium may be other storage media, such as a read-only memory (ROM), a random access memory (RAM), an EPROM, an EEPROM, a magnetic card, a flash memory, an optical card, and a solid state drive (SSD).

What is claimed is:

1. An information processing apparatus comprising a controller including at least one processor, the controller being configured to execute the processing of:
obtaining circumstances information defined as information relating to circumstances around a present location of a user;
determining a recommended exercise as a kind of exercise that is suitable for the circumstances around the present location of the user on the basis of the circumstances information; and
sending suggestion information to a user's terminal used by the user, the suggestion information being information suggesting the user to do the recommended exercise at the present location of the user.

2. An information processing apparatus according to claim 1, wherein the controller obtains at least one of information about an attribute of a place in question defined as a place where the user is presently located, information about a population density of a specific area including the present location of the user, and information about a present date and time as the circumstances information.

3. An information processing apparatus according to claim 2, wherein the controller further executes the processing of:
obtaining an activity pattern of the user; and
determining whether or not circumstances allow the user to do an exercise at the present location on the basis of the activity pattern and the circumstances information,
wherein if it is determined that circumstances do not allow the user to do an exercise at the present location, the controller does not send the suggestion information.

4. An information processing apparatus according to claim 3, wherein if it is conjectured based on the activity pattern, the attribute of the place in question, and the present date and time that the user is taking a rest or waiting for something at the present location, the controller determines that circumstances allow the user to do an exercise at the present location.

5. An information processing apparatus according to claim 3, wherein if it is conjectured based on the activity pattern, the attribute of the place in question, and the present date and time that the user is eating a meal at the present location, the controller determines that circumstances do not allow the user to do an exercise at the present location.

6. An information processing apparatus according to claim 3, wherein if it is conjectured based on the activity pattern, the attribute of the place in question, and the present date and time that the user is taking a bath at the present location, the controller determines that circumstances do not allow the user to do an exercise at the present location.

7. An information processing apparatus according to claim 3, wherein if it is conjectured based on the activity pattern, the attribute of the place in question, the population density of the specific area, and the present date and time that the user is moving by crowded public transportation means, the controller determines that circumstances do not allow the user to do an exercise at the present location.

8. An information processing apparatus according to claim 3, wherein the controller obtains the activity pattern on the basis of a schedule of the user or a record of activities of the user in the past.

9. An information processing apparatus according to claim 2, wherein the controller determines as the recommended exercise a kind of exercise that can be done with only smaller motions when the population density of the specific area is high than when it is low.

10. An information processing apparatus according to claim 1, wherein the controller further obtains effect information and determines the recommended exercise on the basis of the effect information and the circumstances information, the effect information being information about an exercise effect desired by the user.

11. An information processing method comprising the following steps of processing executed by a computer:
a first step of obtaining circumstances information defined as information relating to circumstances around a present location of a user;
a second step of determining a recommended exercise as a kind of exercise that is suitable for the circumstances around the present location of the user on the basis of the circumstances information; and
a third step of sending suggestion information to a user's terminal used by the user, the suggestion information being information suggesting the user to do the recommended exercise at the present location of the user.

12. An information processing method according to claim 11, wherein in the first step, the computer obtains at least one of information about an attribute of a place in question defined as a place where the user is presently located, information about a population density of a specific area including the present location of the user, and information about a present date and time as the circumstances information.

13. An information processing method according to claim 12, wherein the first step includes, in addition to the step of obtaining the circumstances information, the steps of obtaining an activity pattern of the user and the step of determining whether or not circumstances allow the user to do an exercise at the present location on the basis of the activity pattern and the circumstances information, wherein if it is determined that circumstances do not allow the user to do an exercise at the present location, the computer does not execute the third step.

14. An information processing method according to claim 13, wherein if it is conjectured based on the activity pattern, the attribute of the place in question, and the present date and time that the user is taking a rest or waiting for something at the present location, the computer determines that circumstances allow the user to do an exercise at the present location.

15. An information processing method according to claim 13, wherein if it is conjectured based on the activity pattern, the attribute of the place in question, and the present date and time that the user is eating a meal at the present location, the computer determines that circumstances do not allow the user to do an exercise at the present location.

16. An information processing method according to claim 13, wherein if it is conjectured based on the activity pattern, the attribute of the place in question, and the present date and time that the user is taking a bath at the present location, the computer determines that circumstances do not allow the user to do an exercise at the present location.

17. An information processing method according to claim 13, wherein if it is conjectured based on the activity pattern, the attribute of the place in question, the population density of the specific area, and the present date and time that the user is moving by crowded public transportation means, the computer determines that circumstances do not allow the user to do an exercise at the present location.

18. An information processing method according to claim 13, wherein in the step of obtaining the activity pattern, the computer obtains the activity pattern on the basis of a schedule of the user or a record of activities of the user in the past.

19. An information processing method according to claim 11, wherein in the second step, the computer further obtains effect information and determines the recommended exercise on the basis of the effect information and the circumstances information, the effect information being information about an exercise effect desired by the user.

20. A non-transitory storage medium storing an information processing program configured to cause a computer to execute the following steps of processing:
a first step of obtaining circumstances information defined as information relating to circumstances around a present location of a user;
a second step of determining a recommended exercise as a kind of exercise that is suitable for the circumstances around the present location of the user on the basis of the circumstances information; and
a third step of sending suggestion information to a user's terminal used by the user, the suggestion information being information suggesting the user to do the recommended exercise at the present location of the user.
